(19) United States
(12) Patent Application Publication
(10) Pub. No.: US 2025/0255710 A1
(43) Pub. Date: Aug. 14, 2025

(54) EQUINE HEALTH MONITORING DEVICE AND HEALTH PREDICTION SYSTEM

(71) Applicant: Petaluma Technologies Inc., Vancouver (CA)

(72) Inventors: Peter Douglas Whitehead, West Vancouver (CA); Bruce Gregory Mandeville, Columbus, OH (US); Udit Gupta, Surrey (CA)

(21) Appl. No.: 19/051,133

(22) Filed: Feb. 11, 2025

Related U.S. Application Data
(60) Provisional application No. 63/552,292, filed on Feb. 12, 2024.

(51) Int. Cl. A61D 13/00; G16H 50/30

(52) U.S. Cl. CPC A61D 13/00; G16H 50/30

(57) ABSTRACT

An equine health monitoring system generates health predictions based on monitored sensor data from at least one wearable device worn on a leg of an equine. The wearable device includes a pad and one or more securing straps for securing the pad to the leg of the equine. The pad includes an array of spatially distributed sensors for obtaining sensor data and a controller for transmitting the sensor data to a prediction application of a client device. The predication application applies a machine learning model to the sensor data and equine profile to generate likelihood values associated with one or more equine health conditions. The prediction application generates from the likelihood values, a prediction associated with the one or more equine health conditions, and outputs a visual representation of the prediction to a user interface of the client device.

### CROSS-REFERENCE TO RELATED APPLICATIONS

This application claims the benefit of U.S. Provisional Patent Application No. 63/552,292 filed on Feb. 10, 2024, which is incorporated by reference herein.

### BACKGROUND

Monitoring equine health is critical to reducing or preventing effects of injuries or illness in horses. Competitive racehorses are particularly prone to leg injuries, e.g., in their bones, tendons, joints, and ligaments. Failure to detect early warning signs of injuries can ultimately lead to lameness or worse.

### BRIEF DESCRIPTION OF THE DRAWINGS

FIG. 1 is an example embodiment of a computing environment for an equine health monitoring system.

FIG. 2 is a block diagram of a backend server for an equine health monitoring system.

FIG. 3 is a block diagram of a user client for an equine health monitoring system.

FIG. 4A is a first example embodiment of a wearable device for an equine health monitoring system.

FIG. 4B is a second example embodiment of a wearable device for an equine health monitoring system.

FIG. 5 is a diagram illustrating various anatomical structures of an equine leg.

FIG. 6 is an example waveform associated with monitoring temperature data of an equine

FIG. 7A is a first example set of user interfaces associated with a prediction application of an equine health monitoring system.

FIG. 7B is a second example set of user interfaces associated with a prediction application of an equine health monitoring system.

FIG. 7C is a third example set of user interfaces associated with a prediction application of an equine health monitoring system.

FIG. 8 is a set of example waveforms derived from sensor data from a wearable device that may be used to train machine learning models for predicting equine health conditions.

FIG. 9 is another set of example waveforms derived from sensor data from a wearable device that may be used to train machine learning models for predicting equine health conditions

FIG. 10 is a flowchart illustrating an example embodiment of a process for generating predictions about equine health conditions based on sensor data from a wearable device.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

An equine health monitoring system generates health predictions based on monitored sensor data from at least one wearable device worn on a leg of an equine. The wearable device includes a pad and one or more securing straps for securing the pad to the leg of the equine. The pad includes an array of spatially distributed sensors for obtaining sensor data and a controller for transmitting the sensor data to a prediction application of a client device. The predication application applies a machine learning model to the sensor data and an equine profile to generate likelihood values associated with one or more equine health conditions. The prediction application generates from the likelihood values, a prediction associated with the one or more equine health conditions, and outputs a visual representation of the prediction to a user interface of the client device.

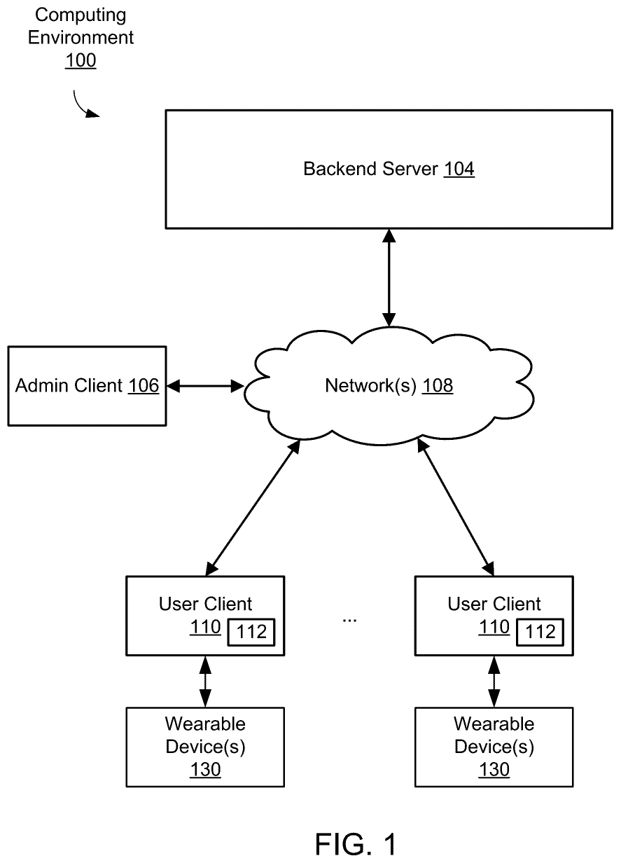

FIG. 1 illustrates an example embodiment of a computing environment 100 for automatically predicting equine health conditions. The computing environment 100 may include a backend server 104, an administrative client 106, one or more user clients 110, one or more wearable devices 130, and a network 108. Alternative embodiments may include different or additional components.

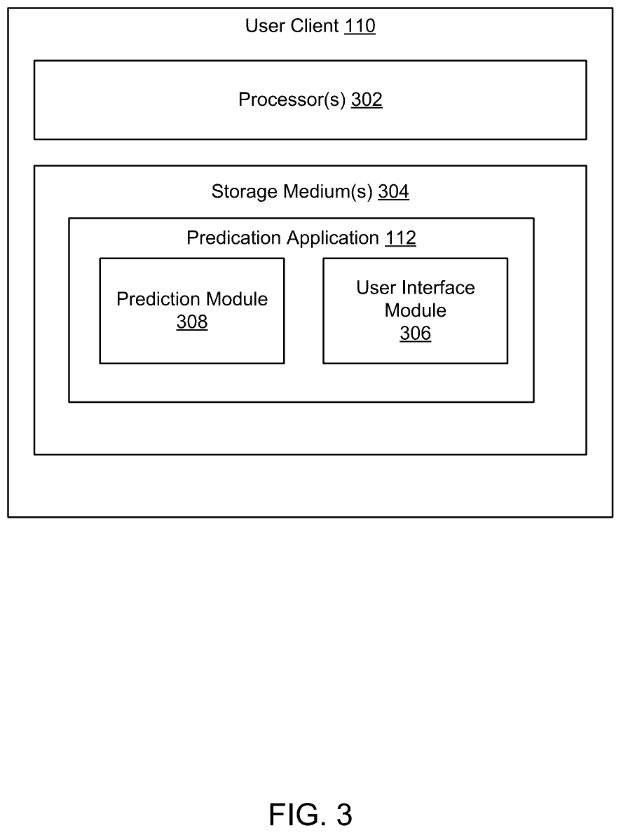

The wearable devices 130 may include a sleeve or boot that wraps around a lower leg of a horse and may be placed adjacent to key tendons, ligaments, bones, joints, or other anatomical targets where injuries tend to occur. The wearable device 130 may include an array of sensors to monitor various conditions in the vicinity of these target areas. For example, sensors may measure temperature, acceleration, orientation changes, pressure, various biometric data, or other sensor data. Some types of sensors may be arranged in sensor arrays to capture highly localized sensor data over a spatial area. For example, in one embodiment of the wearable device 130 includes an array of spatially spaced temperature sensors that each independently monitor temperature at a localized spatial position. Other types of sensors (e.g., inertial sensors) may be placed at a single location on the wearable device 130 instead of in array. The sensors may capture data continuously, periodically, or in response to certain detected events or conditions. Thus, the wearable device 130 may capture one or more sensor data waveforms or arrays of time-varying temperature waveforms, e.g., an array of sensed data (e.g., temperature or other data) measured over time that each corresponds to a localized position on the horse's lower limb. The wearable devices 130 may additionally include various supporting circuitry such as a controller, battery, and transmitter. An example embodiment of a wearable device is illustrated in FIGS. 3A-B described in further detail below.

The user client 110 comprises a computing device capable of communicating with the wearable device 130 and interacting with the backend server 104 via the network 108. The user client 110 may connect with the one or more wearable devices 130 via Bluetooth, WiFi, or any other wireless or wired communication protocol. The wearable device 130 may communicate captured data to the user client 110 in real-time and/or may locally cache data for downloading to the user client 110 at a later time. The user client 110 may furthermore enable sending various configuration settings to the wearable device 130 for calibrating sensors, configurating various control parameters, or other functions.

The user client 110 may enable access to a prediction application 112 that may execute locally as an application installed on the client device 110 or may comprise a web-based application accessible via web browser. The prediction application 112 may include a user interface to enable various data entry that may be processed locally, communicated to the backend server 104, or transferred to the wearable device 130. The user interface may furthermore enable viewing and/or interaction with various information obtained from the backend server 104 and/or the wearable device 130, information derived from local processing of the user client 110, or information directly inputted to the user interface. The user client 110 may furthermore facilitate data transfers between the wearable device 130 and the backend server 104. In various embodiments, the user client 110 may be embodied, for example, as a mobile phone, a tablet, a laptop computer, a desktop computer, a gaming console, a head-mounted display device, or other computing device.

In an embodiment, the user interface may present various tracking data, analytics, and/or health-related recommendations based in part on the monitored biometric data from the wearable device 130. For example, the user interface may present various visualizations (e.g., graphs showing trends over time) of tracked biometric information. The user interface may furthermore track general training activities, periods of rest, diet, or other aspects of equine development, health, and rehabilitation from injury. The user interface may furthermore present inferences relating to current horse health and/or predictions of future horse illness, and/or injury recovery based on the detected biometric data and/or reaction to a drug regime, and other stored profile information for the horse. Predictions may relate to health conditions associated with specific tendons, joints, bones or other anatomical targets. The predictions may furthermore recommend specific training measures such as what activities to adjust, rest, physical therapy, medication, etc. that are predicted to mitigate predicted conditions, prevent further injury, or avoid surgery. The user interface may furthermore recommend and monitor specific warmup regimens that reduce the likelihood of injury.

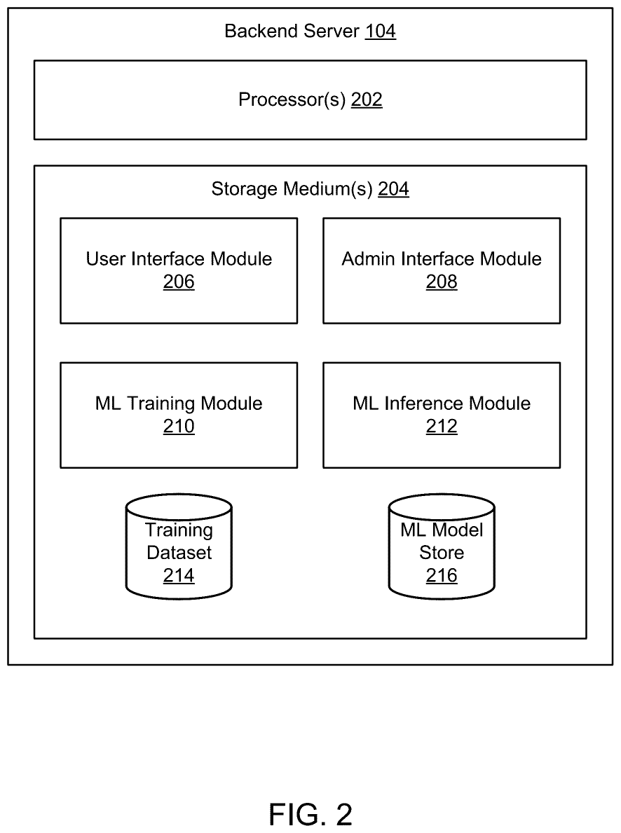

The backend server 104 performs various functions for supporting training of machine learning models, performing inferences based on acquired biometric data or other health-related data, and generating user interface presentations in the user client 110. The backend server 104 may be implemented using cloud processing and storage technologies, on-site processing and storage systems, virtual machines, other technologies, or a combination thereof. For example, in a cloud-based implementation, the backend server 104 may include multiple distributed computing and storage devices managed by a cloud service provider. The various functions attributed to the backend server 104 are not necessarily unitarily operated and managed, and may comprise an aggregation of multiple servers responsible for different functions of the backend server 104 described herein. In this case, the multiple servers may be managed and/or operated by different entities. In various implementations, the backend server 104 may comprise one or more processors and one or more non-transitory computer-readable storage mediums that store instructions executable by the one or more processors for carrying out the functions attributed to the backend server 104 herein. An example embodiment of a backend server 104 is illustrated in FIG. 2 and described in further detail below.

The administrative client 106 comprises a computing device for facilitating administrative functions associated with operation of the backend server 104. For example, the administrative client 106 may comprise a user interface for performing functions such as configuring parameters associated with various machine learning algorithms, initiating deployment of software updates to the user clients 110, etc. The user interface of the administrative client 106 may be embodied as an application installed on the administrative client 106 or may comprise a web-based application accessible via web browser.

The one or more networks 108 provides communication pathways between the backend server 104, the administrative client 106, and/or the user clients 110. The network(s) 108 may include one or more local area networks (LANs) and/or one or more wide area networks (WANs) including the Internet. Connections via the one or more networks 108 may involve one or more wireless communication technologies such as satellite, WiFi, Bluetooth, or cellular connections, and/or one or more wired communication technologies such as Ethernet, universal serial bus (USB), etc. The one or more networks 108 may furthermore be implemented using various network devices that facilitate such connections such as routers, switches, modems, firewalls, or other network architecture.

FIG. 2 is a block diagram illustrating an example embodiment of a backend server 104. The backend server 104 includes one or more processors 202 and one or more storage mediums 204. The one or more storage mediums 204 includes various functional modules (implemented as instructions executable by the one or more processors 202) including a user interface module 206, an administrative interface module 208, a machine learning (ML) training module 210, and an ML inference module 212. The storage medium 204 may furthermore store a training dataset 214 and a model store 216. In alternative embodiments, the backend server 104 may include different or additional modules. The one or more processors 202 and one or more storage mediums 204 are not necessarily co-located and may be distributed (e.g., in a cloud architecture).

The user interface module 206 facilitates server-side functions of a user interface accessible on the user clients 110. In an example operation, the user may input various information via a user interface on the user client 110 that is communicated to the user interface module. Furthermore, the user interface module 206 may output various analytical data, recommendations, or other information pertinent to monitoring equine health.

In an embodiment, the user interface may enable input of various profile information for horses, information about wearable devices, and various other configuration settings. In some embodiments, input data from the user may be obtained interactively by presenting a series of questions via the user interface that enables structured input of data. Questions may be presented for various input forms such as multiple choice, true/false, or text-based inputs. The user interface may utilize various input elements such as radio buttons, drop-down lists, multi-select checkboxes, or freeform text boxes. Here, the user may enter various information about a horse such as physical characteristics (type of horse, weight, age, etc.), health history, training regimen, race history, diet, upcoming events, etc.

The user interface module 206 may furthermore facilitate presentation of various outputs from the backend server 104. For example, the user interface module 206 may output predictions about horse health and recommendations to mitigate effects of strenuous training, stress, lameness, illness, or injury. The user interface module 206 may furthermore present various tracked data relating to user inputs about the horse's diet, training, or other characteristics.

The administrative interface module 208 facilitates various administrative functions associated with operation of the backend server 104. For example, the administrative interface module 208 may present an interface that enables configuration of various parameters of the machine learning module 210 (described below), controls versions and/or access to applications for the user clients 110, or performs other administrative functions.

The training dataset 214 stores various equine health data associated with historical monitoring of equine health. The training dataset 214 may include one or more cloud-based data sources and/or one or more locally accessible data sources. In some implementations, the training dataset 214 may comprise a centralized repository that may aggregate data from multiple different sources. In other implementations, the training dataset 214 may refer to two or more disparate data sources that may be managed by different entities and may be independently accessed by the backend server 104. The training dataset 214 may be accessible via an application programming interface (API) or may enable data to be downloaded via a web browser or other application.

The training dataset 214 may incorporate various structured or unstructured information. For example, structured information may include data organized into predefined fields while unstructured data sources may include articles, notes, or other media. Examples of structured data fields in the training dataset 214 may include, for example, general characteristics of equines (e.g., type of horse, age, location, weight, etc.), characteristics of trainers associated with the horses, general health histories of horses (e.g., lameness, injuries, illness, etc.), diet and training regimens, warmup routines, mitigation steps taken in relation to diagnosed or suspected conditions, race and/or performance history and outcomes, and various biometric monitoring data that may be relevant to analyzing patterns in equine health. The training dataset 214 may furthermore store various contextual information relating to the quality of data, sources of the data, and computation methods. The training dataset 214 may include information from external datasets like weather, humidity etc. linked to timing of any of the above events. The training dataset 214 may be organized into records that are each associated with a specific horse, and include biometric data tracked over time together with observed health information as may be noted by a trainer or groomer, or diagnostics by a veterinarian.

The ML training module 210 trains one or more machine learning models based on a training dataset 214 that includes histories of monitored biometric data (from a wearable device 130) for different horses and their respective health histories. The training dataset 214 may furthermore include profile information for the various horses with tracked biometric data and health histories. The training dataset 214 may encompass data from horses with varying physical characteristics, training regimens, race histories, or other characteristics. Using various machine learning techniques, the ML training module 210 learns relationships between the monitored biometric data, profile information, and health outcomes to enable generation of health-related predictions and recommendations.

In some embodiments, a specific recommendation may be generated based on the predicted health condition. Here, the recommendation may be derived from veterinary and/or training guidelines associated with specific conditions. In other embodiments, recommendations may be derived directly from a machine learning process. Here, the training dataset 214 may include mitigation steps that were taken and associated health outcomes to enable the ML training module 210 to learn relationships between the mitigation steps under different conditions and their effects.

The ML training module 210 may apply a supervised machine learning algorithm to the training dataset 214 to learn a set of model parameters (e.g., weights) for classifying a set of input features. In this implementation, a horse's profile characteristics and tracked biometric sensor data may be labeled in the training dataset 214 with positive and/or negative instances of health outcomes. For example, a set of profile data and tracked biometric data for a horse may be labeled according to instances of lameness, illness, or injury. Labels in the supervised learning approach may furthermore indicate timing of occurrence of the health condition. In various embodiments, the ML models may be trained to infer that a particular health condition is currently present, or one or more ML models may be trained to predict risk that a particular health condition will occur without intervention (e.g., medication, change in training regimen, etc.). In further examples, the ML models may be trained to predict whether a horse has sufficiently warmed up tendons and ligaments prior to ramping up a workout for injury avoidance. In an example implementation, the training module 210 may employ machine learning techniques such as logistic regression, random forest, gradient boosting, or neural networks (such as recurrent neural networks (RNNs), convolutional neural networks (CNNs), etc.). In an embodiment, the training module 210 may periodically retrain the one or more machine learning models as additional training data becomes available.

In other embodiments, an unsupervised learning approach (e.g., clustering) may be employed to train datasets without labels for specific detected health conditions. Here, the ML training module 210 may be trained to detect anomalous conditions where a particular dataset has statistically significant deviations from positive training data (representing normal health conditions). These anomalies may be flagged in the user interface as representing potential health concerns.

The model store 216 stores the one or more machine learning models generated by the training module 210.

The ML inference module 212 applies the one or more machine learning models to an input dataset to generate scores indicative of a likelihood of a health condition being present or occurring in the future. In an embodiment, the inputs to the ML inference module 212 may include stored profile data for a horse and a plurality of sensor data waveforms (that each represent timestamped data from a particular sensor of the biometric device such as temperature over time). For example, the plurality of sensor data waveforms may include an array of sensor data waveforms that each correspond to a different sensor position in the sensor array and are adjacent to different anatomical structures of the horse. In further embodiments, the sensor input data may include tracked IMU data (e.g., tracked acceleration and/or orientation changes) or various other biometric sensor data that may capture data while the horse is in motion.

The inferences may relate to specific health conditions, may relate to specific anatomical targets, and may relate to specific time frames for the condition to occur. For example, a prediction may indicate that lameness associated with a superficial digital flexor tendon has an elevated risk of lameness occurring in the next 10-14 days. The risk scores may comprise the likelihood values (e.g., expressed as a percentage or converted to a score on a predefined scoring scale), a classification of the likelihoods between different risk categories (e.g., low risk/high risk), or a combination thereof. The ML inference module 212 may furthermore generate recommendations for treating or mitigating a health condition. For example, the ML inference module 212 may predict that a period of rest (e.g., 3-5 days) may significantly reduce the likelihood of lameness.

The ML inference module 212 may furthermore generate specific recommendations associated with the health conditions. The recommendations may be derived from the health conditions based on established veterinary, rehabilitation and/or training guidelines, or may be derived from an ML model that predicts the effects of mitigation techniques on health outcomes.

In a further embodiment, the ML inference module 212 may obtain temperature or other sensor data during a warmup routine of the horse and predict, based on the observed data, a likelihood of injury occurring when the horse begins a full workout. Thus, the ML inference module 212 can predict when the horse is sufficiently warmed up. The ML inference module 212 may output recommendations for continuing a warmup routine, completing a warmup routine, or other recommendations to guide training while reducing the likelihood of injuries.

Although various modules of FIG. 2 are illustrated as components of the backend server 104, these modules may be fully or partially implemented in the user device 110 and/or the wearable device 130 in other implementations. For example, all or some of the functions of the user interface module 206 may instead be executed on the user client 110. In this implementation, the user client 110 may download an application from the backend server 104 and the user client 110 may locally execute instructions associated with these functions. Additionally, in some implementations, the user client 110 may include an ML mode store 216 that stores one or more ML models and the user client 110 may locally execute the ML inference module 212 to generate inferences. In this implementation, the ML models may be trained in a manner suitable for edge deployment on a user client 110.

FIG. 3 is a block diagram illustrating an example embodiment of a user client 110. The user client 110 includes one or more processors 302 and one or more storage mediums 304. The one or more storage mediums 304 stores a prediction application 112 (implemented as instructions executable by the one or more processors 302) that comprises at least a user interface module 306 and a prediction module 308. The user client 110 may furthermore include other elements (not shown) of a typical user device, such as a display, an input/output device, a communication interface (e.g., wireless and/or wired interface), etc. The user client 110 may furthermore operate other applications (not shown) such as an operating system, web browsers, or other user applications.

The user interface module 306 facilitates client-side functions of the user interface described herein. For example, the user interface module 306 may enable input of various information and may output various analytical data, recommendations, or other information pertinent to monitoring equine health. The user interface module 306 may operate in conjunction with the user interface module 206 of the backend server 104 or may operate as a standalone application that does not necessarily connect to the backend server 104. Thus, in various implementations, processing tasks may be performed partially or wholly on either the client-side user interface module 306, the server-side user interface module 206, or a combination thereof.

The prediction module 308 generates predictions associated with equine health. In one implementation, the prediction module 308 may facilitate acquisition of the sensor data from the wearable device 130, communication of the sensor data (and/or statistical data derived therefrom) to the ML inference module 212, and obtaining results (e.g., likelihood values, predictions, and/or recommendations) from the ML inference module 212 for presentation on the user client 110. In other implementations, the prediction module 308 may obtain an edge-deployable machine learning model trained on the backend server 104 and may locally apply the machine learning model to sensor data to generate the inferences. In other embodiments, the prediction module 308 may employ a combination of locally generated inferences and inferences obtained from the backend server 104 to generate the predictions.

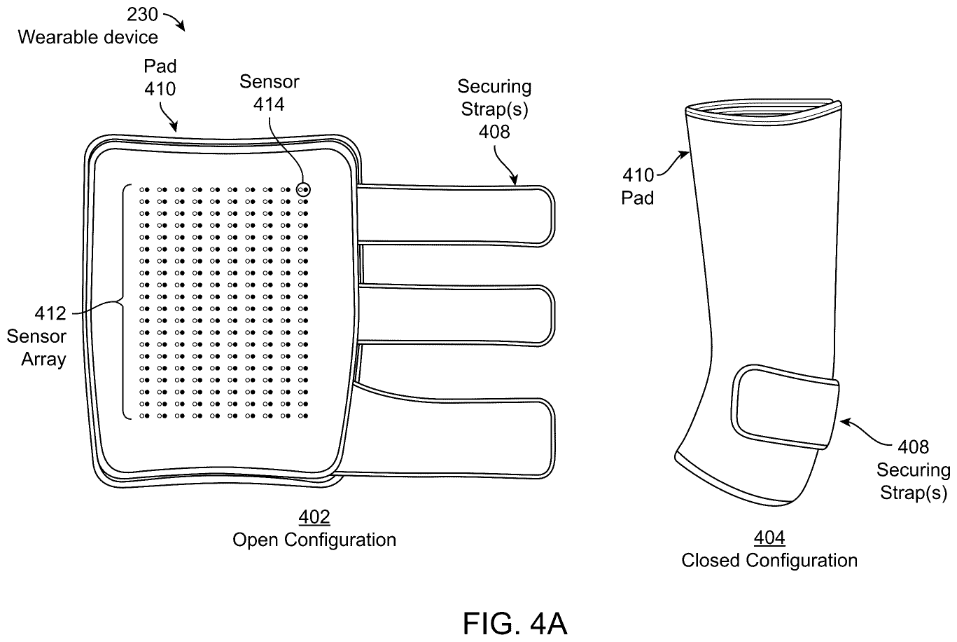

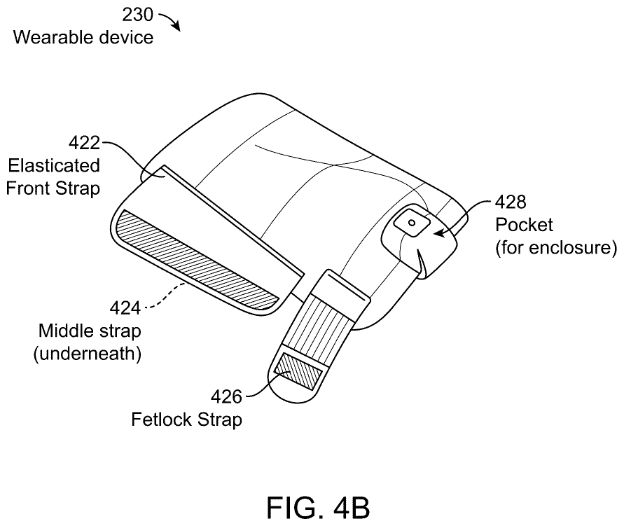

FIGS. 4A-B illustrate example embodiments of a wearable device 130 for obtaining biometric data from a horse. In the example of FIG. 4A, the wearable device 130 comprises a sleeve designed to wrap around the lower limb of a horse and is shown in an open configuration 402 and a closed configuration 404. The device includes a pad 410 with a sensor array 412 (comprising a plurality of sensors 414) and one or more securing straps 408. The pad 410 may comprise a flexible material that enables the sleeve to conform to the physical shape of the limb and provide conformal contact between the sensors 414 of the sensor array 412 and the limb. The sensor array 412 may comprise temperature sensors that each sense temperature at a specific position. For example, one embodiment may include an array 412 of 150-250 temperature sensors 414. In a further embodiment, the sensors 414 could comprise pulse sensors for sensing pulse of the horse. In an embodiment, the pad 410 may include one or more additional sensors such as an inertial measurement unit (IMU) that may measure acceleration and/or change in orientation, blood pressure sensor, pulse sensor, or other biometric sensors.

In the example of FIG. 4B, the device includes an elasticated front strap 422, a middle strap 424, and a Fetlock strap 426 for securing around the limb of the horse. The device furthermore includes a pocket 428 for holding an electronic device that may include various supporting circuitry for the sensor array such as a battery and wireless transmitter.

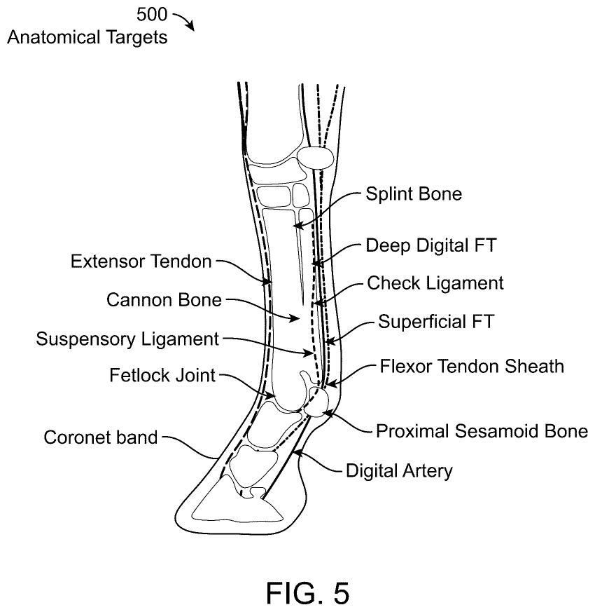

FIG. 5 illustrates a list of anatomical targets 500 such as bones, ligaments, tendons, joints or other anatomical targets 500 that may be monitored by the wearable device 130. As shown, different sensors 414 in the sensor array 412 may be adjacent to different anatomical targets when worn and therefore different sensors 414 or combinations of sensors 414 in the array 412 may implicate temperature, pulse or other biometric data, motion, or other sensor data associated with different monitored targets.

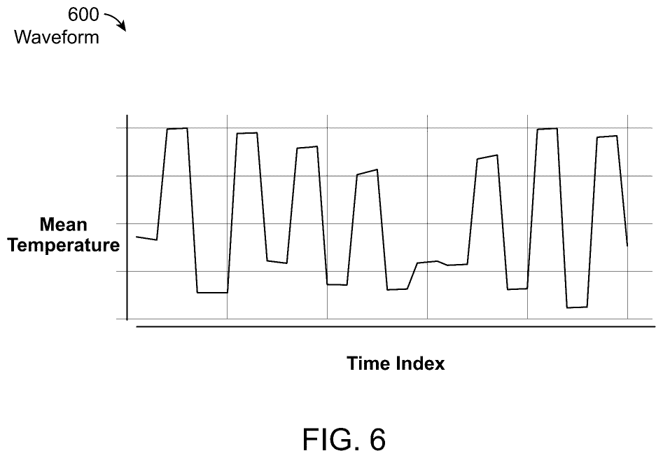

FIG. 6 is an example of a waveform 600 that may be derived from the biometric data obtained from the wearable device 130, and which may be utilized as an input to various machine learning and inference processes described herein. In this example, the x-axis represents a time index (e.g., days or hours) and the y-axis represents a mean temperature across all sensors 414 in the array 412. The example waveform shows rises and falls in measured temperature. In a healthy horse, the rises may generally be expected to occur during training, when the horse is otherwise active, or when the horse is healing. The falls may occur during periods of rest or after therapy modalities. Nevertheless, the temperature waveforms 600 may vary significantly between horses based on training routines, weather, genetics, or other conditions. These differences may affect both baseline temperatures and may affect how temperatures vary under different conditions.

While FIG. 6 shows a waveform representing the mean temperature, other waveforms may similarly be obtained from the monitored sensor data of the wearable device 130 and may be utilized in various machine learning training and inference processes described herein. For example, in one embodiment, waveforms from a sensor array may be aggregated in different ways other than as a mean (e.g., as a median or other function of the individual waveforms). Furthermore, waveforms may be obtained for each individual sensor 414 in an array 412 that are not necessarily aggregated together. In further embodiment, machine learning processes may operate on sensor waveforms representing other types of sensor data such as inertial data, pressure data, pulse data, or other biometric data from either individual sensors or data aggregated across a sensor array.

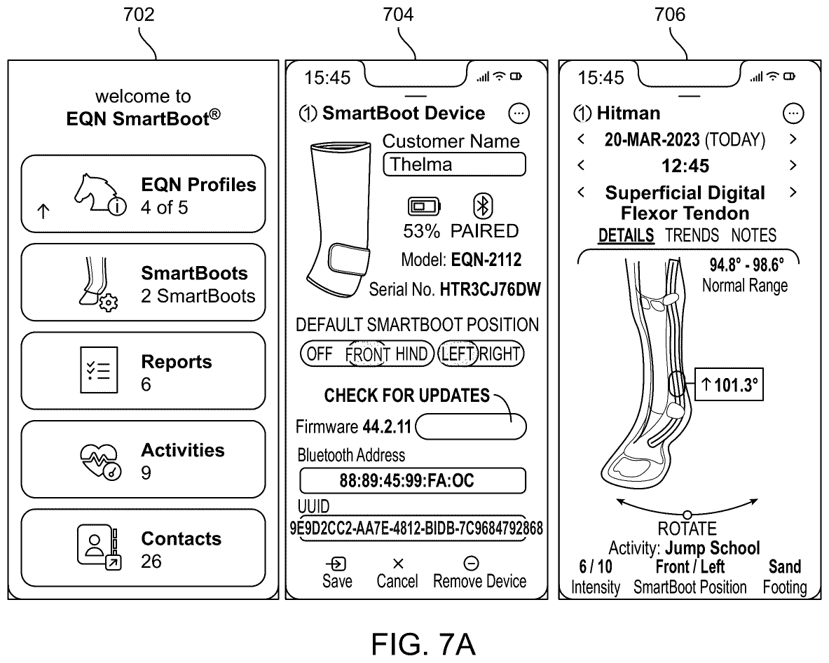

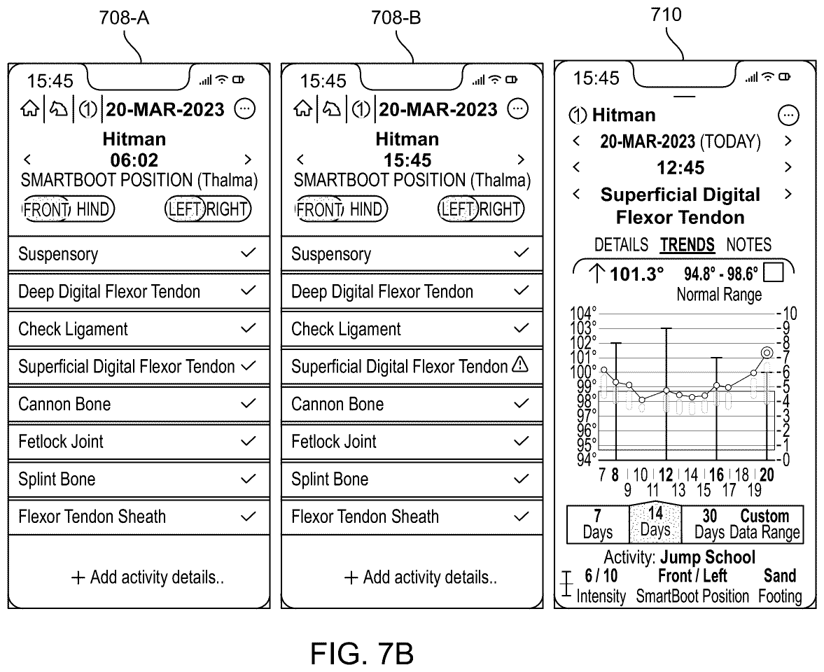

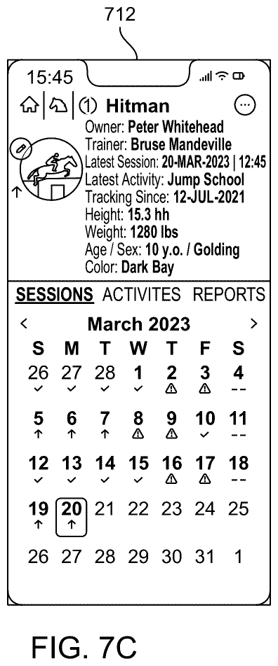

FIGS. 7A-C illustrate a set of example user interfaces that may be presented on the user client 110 in association with the prediction application 112. In a first example interface 702, a user is presented with a home screen that enables access to various functions of the application. For example, the interface 702 presents menu items for setting up profiles for horses, connecting one or more wearable devices 130 and associating the wearable devices 130 with a particular horse, viewing or generating reports, monitoring horse activities, and viewing or editing contacts.

A second example interface 704 enables pairing and configuration of a wearable device 130. For example, this interface 704 may enable the user to pair the wearable device 130 (e.g., via Bluetooth) to the user client 110, associate the wearable device 130 with a particular profile for a horse, and select a position on the horse where the wearable device 130 will be placed (e.g., front or hind and left or right).

A third example interface 706 presents monitoring data associated with activities of the horse. In this example, the interface 706 indicates that an elevated temperature is detected in the superficial digital flexor tendon. The interface 706 may enable the user to switch between different anatomical structures (such as those shown in FIG. 4), like bones, joints, or tendons of the horse to view respective monitoring data, view trends over time, and record or view notes relating to equine health.

A fourth example interface 708 (where 708-A, 708-B represent different partial views of scrollable list) shows a list of anatomical targets (e.g., tendons, ligaments, bones, joints, etc.) that may be monitored. In this example, a checkmark or green indicator indicates measured biometric data in the normal range and an exclamation or yellow or red icon indicates an abnormality.

A fifth example interface 710 presents biometric monitoring trends. This example interface shows temperature for a superficial digital flexor tendon over a 14-day period displayed in a visual graph. The graph illustrates an abnormal rise in temperature occurring over the last several days. The rise may be indicative of a potential injury or illness associated with this tendon.

A sixth example interface 712 illustrates monitoring data presented in a calendar view. The calendar view may indicate when biometric data shows normal health, elevated temperatures, and/or abnormal temperatures.

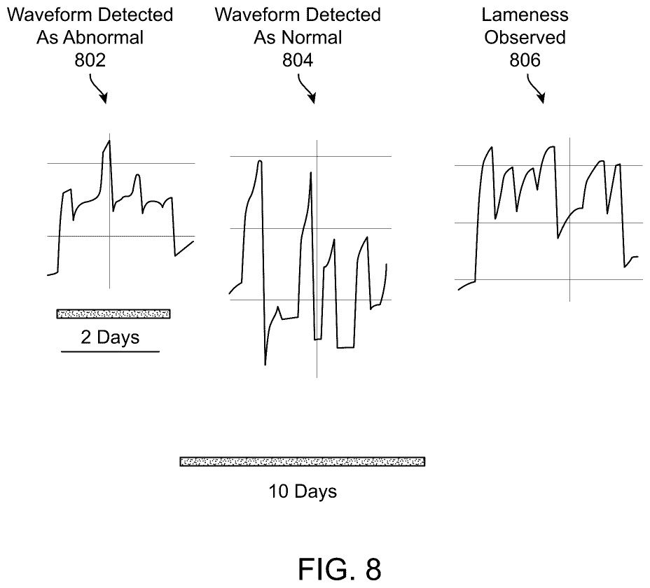

FIG. 8 illustrates additional example waveforms 800 and associated user interfaces 850 that can be generated using the techniques described herein. In this example, the monitoring trend shows abnormal biometric readings for a two day period, followed by normal readings for a 10-day period, followed by observations of lameness. This example illustrates that lameness may occur well after early signs are present and while there is still time to take intervening measures.

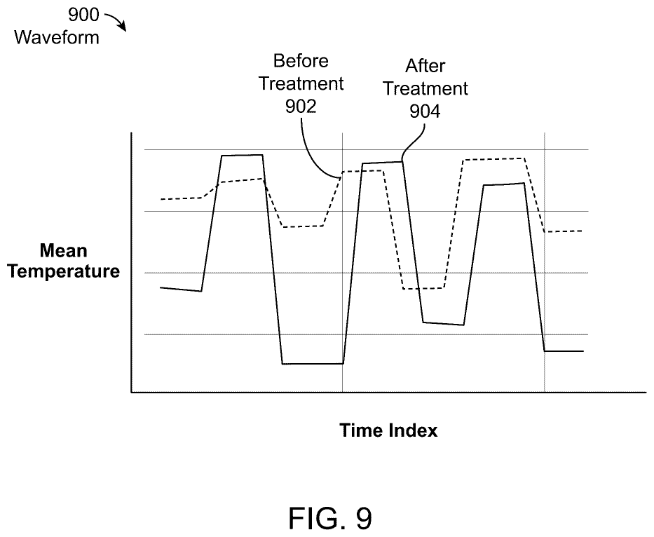

FIG. 9 illustrates additional example waveforms 900 that compare mean temperature for a horse in the three days before starting treatment 902 with anti-inflammatory therapeutic (phenylbutazone in this example) and three days after treatment 904. The waveforms 902, 904 represent different three-day periods, but are overlaid on top of each other fur the purpose of visual comparison. These waveforms 902, 904 may be presented in the user interface of the prediction application 112 to enable a caretaker to monitor effects of treatment. Additionally, this waveform data may be used to train machine learning models to learn the effects of different therapeutics on the horse's health and may enable an ML inference module 212 to generate predictions relating to these effects. For example, the machine learning model may predict how a horse will respond to a specific therapeutic based on the waveforms monitored prior to treatment 902 and other characteristics of the horse. Different models may be learned for different possible treatments to enable the models to recommend one or more treatments in view of the observed conditions.

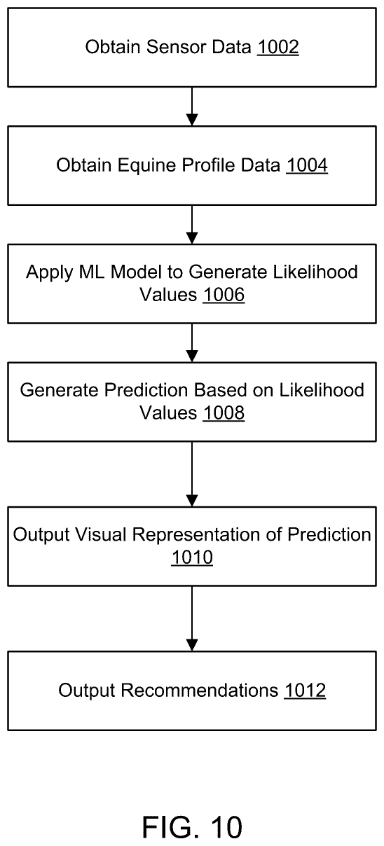

FIG. 10 is a flowchart illustrating an example embodiment of a process for generating equine health predictions based on monitored sensor data from at least one wearable device 130 worn on a leg of an equine. A prediction application 112 obtains 1002 sensor data from the wearable device 130, which includes an array of biometric data waveforms corresponding to different spatially distributed sensor locations. For example, the array of biometric data waveforms may comprise an array of temperature data waveforms from temperature sensors spatially distributed in the wearable device 130 that measure temperature at respective locations of the leg. In other embodiments, the sensor data may include accelerometer data, gyroscope data, motion data, pulse data, and/or pressure data.

The prediction application 112 also obtains 1004 equine profile data associated with the equine. The equine profile data may include various characteristics of the equine that may affect health predictions and may also include information about the placement and type of wearable device 130. For example, the equine profile data may include position data indicative for the at least one wearable device selected between a left front leg, a right front leg, a left hind leg, and a right hind leg. The equine profile data may also include information such as an age of the equine, a type of the equine, genetic information about the equine, an activity history of the equine, a future activity plan for the equine, a health history of the equine, a weight of the equine, a height of the equine, an age of the equine, a gender of the equine, an identification of a trainer of the equine, an identification of an owner of the equine, and/or an ancestry history of the equine.

The prediction application 112 applies 1006 a machine learning model to the equine profile data and the sensor data to generate likelihood values associated with one or more equine health conditions. In one implementation, the machine learning model may be derived from a supervised learning algorithm that learns relationships between training sensor waveforms and diagnosed health conditions. Other machine learning models may be trained based on an unsupervised learning algorithm that learns patterns of training sensor data associated with normal health conditions and enables the machine learning model to detect anomalous patterns in the sensor data. The likelihood values may include separate likelihood values associated with one or more specific anatomical targets of the leg such as a splint bone, a deep digital flexor tendon, a check ligament, a superficial flexor tendon, a flexor tendon sheet, a proximal sesamoid bone, a digital artery, an extensor tendon, a cannon bone, a suspensory ligament, a fetlock joint, a coronet band, and/or other anatomical structures. The likelihood values may represent predictions of conditions such as lameness, illness, sprain, tendonitis, and fracture affecting the leg generally or specific to one or more anatomical targets. The likelihood values may furthermore represent predictions associated with one or more future time frames. For example, the likelihood values may indicate a prediction of condition presenting itself within three days, within a week, within two weeks, or within some other time frame.

The predication application then generates 1008, from the likelihood values, a prediction associated with the one or more equine health conditions. The prediction may indicate a predicted anatomical target (e.g., a specific leg and/or one or more specific anatomical structures of the leg that is affected), a predicted health condition associated with the anatomical target, and a predicted timeframe associated with presentation of the predicted health condition of the anatomical target. For example, a prediction may indicate that an equine is at risk of experiencing lameness in the front right leg within the next two weeks. In one implementation, one or more likelihood values may be compared to a threshold (or some other comparison criteria) to determine if a risk level is satisfied. In other implementations, a stratified risk level may be generated and output based on the likelihood values (e.g., low risk, medium risk, high risk). In further embodiments, one or more likelihood values may be directly output in the prediction (e.g., expressed as a risk percentage).

The predication application outputs 1010 a visual representation of the prediction to a user interface of the client device. For example, the prediction may be outputted as text, in graphical form (e.g., as a color-coded and/or labeled diagram of the horse's leg), or in one or more charts, graphs, or other visual representations. In an embodiment, the prediction application 112 may also output a visual representation (e.g., text, graphs, charts, etc.) of the various sensor data, likelihood values, or statistical data derived therefrom together with the prediction.

Furthermore, the predication application may output 1012 one or more recommendations for mitigating the one or more equine health conditions in the prediction. For example, recommendations may include stopping or modifying a training regime, seeking physical therapy, consulting a veterinarian regarding medication, or other treatment plans.

The prediction application 112 may also employ the machine learning techniques described herein to generate predictions about effectiveness of a proposed treatment. Here, a machine learning model may be trained to learn how particular treatments correlate to changes in sensor readings during or after the treatment, which may be indicative of effectiveness of treatment. Using this type of model, the prediction application 112 may generate one or more predicted future sensor data waveforms from application of the machine learning model, and determine the likelihood values based on the predicted future sensor data waveforms. For example, the machine learning model may predict a future set of sensor readings based on a history of sensor measurements, and then derive predictions about associated health conditions from those future predicted sensor data. Here, predictions may be generated with and without a specific treatment being provided and the likelihood values may be indicative of likelihood of success in the treatment resolving the health condition.

The described embodiments incorporate multiple technical improvements that improve the functioning of computer systems, machine learning techniques, data management systems (particularly as related to sensor data management), computer-based user interfaces, wearable devices, and other technologies and technical fields. For example, the disclosed embodiments improve data availability by generating predictions about health conditions from sensor data of a wearable device. By distilling large amounts of sensor data to useful predictions, the disclosed embodiments effectively compress the large amounts of sensor data, thereby conserving network bandwidth and storage resources relative to conventional manual data analysis techniques. The described embodiments also relate to an improvement in the field of equine wearable devices by incorporating sensors that can detect fine-grained changes in conditions over a spatial area targeting anatomical structures at high levels of specificity. This data may be seamlessly communicated and presented on a client device with high accuracy and predictive power relative to conventional techniques.

The described systems and methods also provide a technical improvement in the field of equine medical treatment and diagnosis methods. The improved monitoring methods enable detection of health conditions and intervening treatments not previously available. For example, the predictions may identify statistical trends in large amounts of data that can detect correlations between monitored data and health conditions in ways that are not conventionally identifiable to human practitioners.

Embodiments of the described computing environment 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A system comprising:
at least one wearable device comprising:
a pad for positioning adjacent to a leg of an equine;
one or more securing straps for securing the pad to the leg of the equine;
an array of spatially distributed sensors on the pad, the array of spatially distributed sensing for sensing sensor data including an array of biometric data pertaining to the leg of the equine; and
a controller for obtaining the sensor data and transmitting the sensor data to a client device; and
a non-transitory computer-readable storage medium storing instructions for generating equine health predictions based on the sensor data from at least one wearable device worn on a leg of an equine, the instructions when executed by one or more processors of the client device causing the one or more processors to perform steps comprising:
obtaining by a client device from the at least one wearable device worn on the leg of the equine, the sensor data including the array of biometric data waveforms corresponding to different spatially distributed sensor locations;
obtaining equine profile data associated with the equine;
applying a machine learning model to the equine profile data and the sensor data to generate likelihood values associated with one or more equine health conditions;
generating from the likelihood values, a prediction associated with the one or more equine health conditions; and
outputting a visual representation of the prediction to a user interface of the client device.

2. The system of claim 1, wherein the array of spatially distributed sensors comprises an array of temperature sensors for sensing an array of temperature data waveforms.

3. The system of claim 1, wherein the wearable device further comprises at least one additional sensor, and wherein the sensor data includes at least one of: accelerometer data, gyroscope data, motion data, pulse data, and pressure data.

4. A method for generating equine health predictions based on monitored sensor data from at least one wearable device worn on a leg of an equine, the method comprising:
obtaining by a client device from the at least one wearable device worn on the leg of the equine, sensor data including an array of biometric data waveforms corresponding to different spatially distributed sensor locations;
obtaining equine profile data associated with the equine;
applying a machine learning model to the equine profile data and the sensor data to generate likelihood values associated with one or more equine health conditions;
generating from the likelihood values, a prediction associated with the one or more equine health conditions; and
outputting a visual representation of the prediction to a user interface of the client device.

5. The method of claim 4, wherein the array of biometric data waveforms comprises an array of temperature data waveforms.

6. The method of claim 4, wherein the sensor data furthermore includes at least one of: accelerometer data, gyroscope data, motion data, pulse data, and pressure data.

7. The method of claim 4, wherein the equine profile data includes position data indicative for the at least one wearable device selected between a left front leg, a right front leg, a left hind leg, and a right hind leg.

8. The method of claim 4, wherein the equine profile data includes an age of the equine, a type of the equine, genetic information about the equine, an activity history of the equine, a future activity plan for the equine, a health history of the equine, a weight of the equine, a height of the equine, an age of the equine, a gender of the equine, an identification of a trainer of the equine, an identification of an owner of the equine, and an ancestry history of the equine.

9. The method of claim 4, wherein the likelihood values associated with the one or more equine health conditions include likelihood values associated with each of a set of anatomical targets of the leg.

10. The method of claim 9, wherein the set of anatomical targets includes at least one of: a splint bone, a deep digital flexor tendon, a check ligament, a superficial flexor tendon, a flexor tendon sheet, a proximal sesamoid bone, a digital artery, an extensor tendon, a cannon bone, a suspensory ligament, a fetlock joint, and a coronet band.

11. The method of claim 4, wherein the likelihood values associated with the one or more equine health conditions includes likelihood of values associated with one or more of lameness, illness, sprain, tendonitis, and fracture.

12. The method of claim 4, wherein the likelihood values associated with the one or more equine health conditions include likelihood values associated with a plurality of future time frames indicative of when the equine presents the one or more equine health conditions.

13. The method of claim 4, wherein the prediction includes at least one of: a predicted anatomical target, a predicted health condition associated with the anatomical target, and a predicted timeframe associated with presentation of the predicted health condition of the anatomical target.

14. The method of claim 4, further comprising generating from at least one of the sensor data, the likelihood values, and the prediction, a recommendation for mitigating the one or more equine health conditions.

15. The method of claim 4, wherein the machine learning model is trained according to a supervised learning algorithm to learn relationships between training sensor waveforms and diagnosed health conditions.

16. The method of claim 4, wherein the machine learning model is trained according to an unsupervised learning algorithm to learn patterns of training sensor data associated with normal health conditions and to enable the machine learning model to detect anomalous patterns in the sensor data.

17. The method of claim 4, wherein the machine learning model is trained from training sensor data obtained from equines before treatment for a health condition and after the treatment for the health condition, and wherein applying the machine learning model to generate the likelihood values comprises:
generating from the machine learning model, one or more predicted future sensor data waveforms; and
determining the likelihood values based on the predicted future sensor data waveforms, wherein the likelihood values are indicative of likelihood of success in resolving the health condition.

18. The method of claim 4, further comprising:
generating a visual representation of the sensor data; and
presenting the visual representation of the sensor data in the user interface.

19. A non-transitory computer-readable storage medium storing instructions for generating equine health predictions based on monitored sensor data from at least one wearable device worn on a leg of an equine, the instructions when executed by one or more processors causing the one or more processors to perform steps comprising:
obtaining by a client device from the at least one wearable device worn on the leg of the equine, sensor data including an array of biometric data waveforms corresponding to different spatially distributed sensor locations;
obtaining equine profile data associated with the equine;
applying a machine learning model to the equine profile data and the sensor data to generate likelihood values associated with one or more equine health conditions;
generating from the likelihood values, a prediction associated with the one or more equine health conditions; and
outputting a visual representation of the prediction to a user interface of the client device.

20. The non-transitory computer-readable storage medium of claim 19, wherein the array of biometric data waveforms comprises an array of temperature data waveforms.
